(19) United States
(12) Patent Application Publication
(10) Pub. No.: US 2002/0058996 A1
(43) Pub. Date: May 16, 2002

(54) CORNEAL REFRACTIVE PROPERTIES ALTERING DEVICE AND METHOD

(76) Inventors: THOMAS A. SILVESTRINI, ALAMO, CA (US); MARK L. MATHIS, FREMONT, CA (US); JOHN A. SCHOLL, DANVILLE, CA (US); ROBERT A. PROUDFOOT, SANTA CLARA, CA (US)

Correspondence Address: CHRIS JAMES, Kera Vision, Inc., 48630 Milmont Drive, Fremont, CA, 94538-7353, US

(21) Appl. No.: 08/827,624

(22) Filed: Apr. 9, 1997

Related U.S. Application Data
(63) Continuation of application No. 08/437,415, filed on May 12, 1995, now abandoned.
Application No. 08/437,415 is a continuation-in-part of application No. 08/399,300, filed on Mar. 2, 1995, now abandoned.

(51) Int. Cl. A61F 2/14

(52) U.S. Cl. 623/5.12

(57) ABSTRACT

A device and method for refractive correction of the eye in order to improve the vision of the eye. The device is an intrastromal corneal ring (ICR) where the cone angle θ is varied according to the desired change in corneal refractive properties. The method involves the insertion of the ICR with appropriate cone angle for the desired change those properties.

This application is a continuation of application Ser. No. 08/437,415, filed May 12, 1995, now abandoned, which is a continuation in part of application Ser. No. 08/399,300 filed Mar. 2, 1995, now abandoned.

### DESCRIPTION

1. Technical Field

The present invention relates to a device and method for changing corneal refractive properties including the radius of curvature and/or the aspheric shape of the cornea of an eye. More specifically, the invention involves an intrastromal corneal ring having a cone angle or multiple cone angles which effect this change when the intrastromal corneal ring is inserted into the cornea, and the method for effecting that change.

2. Background

Anomalies in the shape of the eye can cause visual disorders. Axial hyperopia (“farsightedness”) occurs when the front-to-back distance in the eyeball is too small. Curvature hyperopia occurs when the corneal curvature is less than normal and therefore is flatter than the normal cornea. In these cases, parallel rays originating greater than 20 feet from the eye focus behind the retina. In contrast, when the front-to-back distance of the eyeball is too large, axial myopia (“nearsightedness”) occurs. When the corneal curvature is too great, curvature myopia occurs. In these cases, the focus of parallel rays entering the eye occurs in front of the retina. Astigmatism is a condition which occurs when the parallel rays of light do not focus to a single point within the eye, but rather have a variable focus due to the fact that the corneal curvature varies in different meridians. Light is therefore refracted different distances and focuses at different regions. Some degree of astigmatism is normal, but where astigmatism is too pronounced, it must often be corrected. Presbyopia is an age-related condition that results in the loss of the ability of the eye to change focal length.

Hyperopia, myopia, presbyopia and astigmatism are usually corrected by glasses or contact lenses. Surgical methods for the correction of such disorders have been cited in the literature and include radial keratotomy (see e.g. U.S. Pat. Nos. 4,815,463 and 4,688,570) and laser corneal ablation (see e.g. U.S. Pat. No. 4,941,093). Another method for correcting those disorders is through implantation of polymeric rings in the eye's corneal stroma to change the curvature of the cornea. Previous work involving the implantation of polymethylmethacrylate (PMMA) rings, allograft corneal tissue and hydrogels is well documented. One of the ring devices involves a ring design that allows a split ring to be inserted into a channel dissected in the stromal layer of the cornea. The device uses a minimally invasive incision through which the channel for the implant is created and through which the implant is inserted and adjusted. Adjustment of the device normally involves an adjustment of ring size or diameter.

U.S. Pat. No. 4,452,235 describes a method and apparatus for corneal curvature adjustment. The method involves inserting one end of a split end adjusting ring into the cornea of the eye and moving the ring in a circular path until its ends meet. The ends are thereafter adjusted relative to each other until the shape of the eye has assumed a desired curvature whereupon the ends are fixedly attached to maintain the desired curvature of the cornea.

PCT/US93/00059, the entirety of which is incorporated by reference, describes a method for the refractive correction of the eye as well. Intrastromal corneal rings of varying thickness are inserted into the corneal stroma to change the curvature of the cornea.

The present invention involves the use of intrastromal corneal rings of varying cone angles to change the curvature of the cornea for the refractive adjustment of the eye.

### SUMMARY OF THE INVENTION

The present invention involves changing the configuration of the cornea as a function of cone angle. According to the present invention, an intrastromal corneal ring is provided with a mismatching cone angle selected to independently impart a force on the corneal tissue when the intrastromal corneal ring is positioned at the desired location in the cornea. More specifically, the mismatching cone angle can independently effect a change in the radius of curvature and/or the aspheric shape of the cornea. Thus, the cone angle is chosen based on the starting curvature of the eye, the thickness of the intrastromal corneal ring and the type of corneal curvature and/or aspheric change desired. The cone angle may be selected to (1) maintain the surface of the eye close to aspheric shape of the eye prior to insertion of the ring or (2) alter the aspheric shape of the eye as desired, for example. In sum, the invention involves providing an intrastromal corneal ring having a mismatching cone angle and changing the refractive properties of an eye using an intrastromal corneal ring having a mismatching cone angle.

According to the present invention, a mismatching cone angle preferably is described with reference to an imaginary intrastromal corneal ring superimposed on the insertion site prior to insertion. This permits calculation of the appropriate angle with reference to the cornea before its configuration is changed through the insertion of the intrastromal corneal ring. Thus, with reference to such an imaginary intrastromal corneal ring having a mismatching cone angle, the major axis of substantially any radial, transverse cross-section of the intrastromal corneal ring would not be parallel to a line in the same plane as that major axis and tangent to the anterior surface of the cornea at the point where the line that bisects the major axis line (defined as the line extending along the major axis and bounded by the outer surface of the intrastromal corneal ring) and is perpendicular thereto, intersects the anterior surface of the cornea.

A mismatching cone angle also can be described relative to an equation that for a given D_{cc}, Rᵢ and d, describes a matching cone angle. For a given D_{cc}, Rᵢ, and d, a cone angle which corresponds to a matching cone angle can be described according to the following equation:

$$θ = {sin}^{- 1} \frac{{D}_{cc}}{2 ( {R}_{i} - d )} , where \qquad ( 1 )$$

θ=cone angle

D_{cc}=diameter of the intrastromal corneal ring (center to center where each center is defined as the midpoint of the line extending across a radial, transverse section of the intrastromal corneal ring and on the major axis thereof)

Rᵢ=initial corneal radius of curvature (i.e., before implanting the intrastromal corneal ring)

d=depth of the intrastromal corneal ring in the cornea measured radially from the anterior corneal surface to the midpoint of a radial line, extending across the thickest or largest radial dimension of the above-referenced radial, transverse section of the intrastromal corneal ring and bounded thereby.

Thus, for a given D_{cc}, Rᵢ and d, a mismatching cone angle is one that does not equal the cone angle described in the foregoing equation.

An alternate equation for describing a matching cone angle preferably is used to account for intrastromal corneal ring thickness when intrastromal corneal rings with relatively large thicknesses are used. This is generally preferred when accounting for thicknesses above about 0.15 mm (i.e., thicknesses that provide sufficient thickness to flatten the cornea independent of cone angle). According to this refined equation:

$$θ = {sin}^{- 1} \frac{{D}_{cc}}{2 [ ( {R}_{i} - d ) + \vert Δ {R}_{t} \vert ]} , where \qquad ( 2 )$$

ΔRₜ=the expected change induced by intrastromal corneal ring thickness independent of cone angle.

Thus, when accounting for such thickness, a mismatching cone angle is one that does not equal the cone angle described in equation (2) for a given D_{cc}, Rᵢ, d and ΔRₜ.

According to a preferred method for changing the refractive characteristics of an eye, the method comprises the steps of: (a) providing a group of intrastromal corneal rings having different cone angles; (b) determining an amount of corrective refraction desired; (c) selecting an intrastromal corneal ring from the group of intrastromal corneal rings based on the amount of corrective refraction determined in step (b); and (d) inserting the intrastromal corneal ring selected in step (c) into the cornea of the eye.

With this method, the corneal curvature can be changed by using intrastromal corneal rings with different cone angles. In addition, if a desired amount of refractive correction has not been achieved, the inserted intrastromal corneal ring can be removed and a second intrastromal corneal ring from the group and having a different cone angle implanted.

More specifically, the second intrastromal corneal ring can be selected to have a cone angle greater than the cone angle of the intrastromal corneal ring implanted in step (c) if the eye or cornea does not flatten by the desired amount to treat myopia, for example.

Alternatively, the second intrastromal corneal ring can be selected to have a cone angle less than the cone angle of the intrastromal corneal ring implanted in step (c) if the region of the cornea inside the intrastromal corneal ring does not steepen by the desired amount. This generally would be the case when treating a hyperopic condition.

In addition, a number of the intrastromal corneal rings provided in step (a) can be provided with different thicknesses and/or diameters to accommodate a number of different corneal configurations. For example, a number of the intrastromal corneal rings can have the same outer diameter but differ in cone angle.

According to a further embodiment of the invention, an intrastromal corneal ring is constructed with multiple cone angles. That is, the cone angle changes along the circumferential direction of the intrastromal corneal ring. This construction is particularly advantageous for treating astigmatism (or astigmatism concurrent with either myopia or hyperopia) where the corneal curvature varies in different meridians. For example, the cone angles can be selected to change the corneal curvature such that the light rays tend more to focus to a single point.

The above is a brief description of some of the advantages of the present invention. Other features, advantages and embodiments of the invention will be apparent to those skilled in the art from the following description, accompanying drawings and appending claims.

### BRIEF DESCRIPTION OF THE DRAWINGS

FIG. 1 is a schematic representation of a horizontal section of the eye.

FIG. 2 is a schematic illustration of the anterior portion of the eye showing the various layers of the cornea.

FIG. 3 is a schematic representation of an eye showing the average corneal curvature radius and aspheric shape of the cornea.

FIG. 4 is a schematic representation of a hyperopic eye showing the average corneal curvature radius and the aspheric shape of the cornea.

FIG. 5 is a top plan view of one embodiment of an intrastromal corneal ring according to the present invention.

FIG. 6 is a sectional view of the intrastromal corneal ring of FIG. 5 taken along lines 6A-6A.

FIG. 7A is a cross-sectional view of a cornea showing an intrastromal corneal ring positioned therein and having a cross-sectional shape that is the same as the intrastromal corneal ring of FIG. 6 and a matching cone angle.

FIG. 7B is a diagrammatic view of the intrastromal corneal ring of FIG. 7A illustrating geometric relationships relative to a cornea.

FIG. 8 is a cross-sectional view of a cornea showing an intrastromal corneal ring having a cross-sectional shape that is the same as that shown in FIG. 7 but with an active or mismatching cone angle that according to one embodiment of the invention effects the flattening of the corneal anterior surface for treating myopia.

FIG. 9 is a cross-sectional view of a cornea showing an intrastromal corneal ring having a cross-sectional shape that is the same as that shown in FIG. 7 but with an active or mismatching cone angle that according to another embodiment of the invention effects the steepening of the corneal anterior surface for treating hyperopia.

FIGS. 10 and 11 illustrate geometric relationships between an active or mismatching intrastromal corneal ring and a cornea in accordance with the present invention.

FIGS. 12A, 12B, 12C, 12D and 12E are graphs showing the relationship among intrastromal corneal ring stiffness, correction in diopters and cone angle for a given intrastromal corneal ring and two different stiffnesses.

FIG. 13 illustrates a further embodiment of the intrastromal corneal ring of the present invention in which one intrastromal corneal ring is provided with multiple cone angles.

FIG. 14 is a sectional view taken along line 14-14 in FIG. 13.

FIG. 15 is a sectional view taken along line 15-15 in FIG. 13.

Like elements in the drawings bear the same reference numerals.

### MODES FOR CARRYING OUT THE INVENTION

Prior to explaining the details of the inventive devices and methods, an explanation of the physiology of the eye will be provided. Referring to FIG. 1, a horizontal section of the eye is shown. Globe 11 of the eye resembles a sphere with an anterior bulged spherical portion representing cornea 12.

Globe 11 of the eye consists of three concentric coverings enclosing the various transparent media through which the light must pass before reaching the sensitive retina (18). The outermost covering is a fibrous protective portion the posterior five-sixths of which is white and opaque and called the sclera (13), and sometimes referred to as the white of the eye where visible to the front. The anterior one-sixth of this outer layer is the transparent cornea (12).

A middle covering is mainly vascular and nutritive in function and is comprised of the choroid 14, ciliary body 16 and iris 17. Choroid 14 generally functions to maintain retina 18. Ciliary body 16 is involved in suspending lens 21 and accommodation of the lens. Iris 17 is the most anterior portion of the middle covering of the eye and is arranged in a frontal plane. It is a thin circular disc corresponding to the diaphragm of a camera, and is perforated near its center by a circular aperture called the pupil (19). The size of the pupil varies to regulate the amount of light which reaches retina 18. It contracts also to accommodation, which serves to sharpen the focus by diminishing spherical aberration. Iris 17 divides the space between cornea 12 and lens 21 into an anterior chamber 22 and a posterior chamber 23. The innermost portion of covering is retina 18, consisting of nerve elements which form the true receptive portion for visual impressions.

Retina 18 is a part of the brain arising as an outgrowth from the fore-brain, with optic nerve 24 serving as a fiber tract connecting the retina part of the brain with the fore-brain. A layer of rods and cones, lying just beneath a pigmented epithelium on the anterior wall of the retina serve as visual cells or photoreceptors which transform physical energy (light) into nerve impulses.

Vitreous body 26 is a transparent gelatinous mass which fills the posterior four-fifths of globe 11. At its sides it supports ciliary body 16 and retina 18. A frontal saucer-shaped depression houses the lens.

Lens 21 of the eye is a transparent bi-convex body of crystalline appearance placed between iris 17 and vitreous body 26. Its axial diameter varies markedly with accommodation. Ciliary zonule 27, consisting of transparent fibers passing between ciliary body 16 and lens 21 serves to hold lens 21 in position and enables the ciliary muscle to act on it.

Referring again to cornea 12, this outermost fibrous transparent coating resembles a watch glass. Its curvature is somewhat greater than the rest of the globe and is ideally spherical in nature. However, often it is more curved in one meridian than another giving rise to astigmatism. A central third of the cornea is called the optical zone with a slight flattening taking place outwardly thereof as the cornea thickens towards its periphery. Most of the refraction of the eye takes place through the cornea.

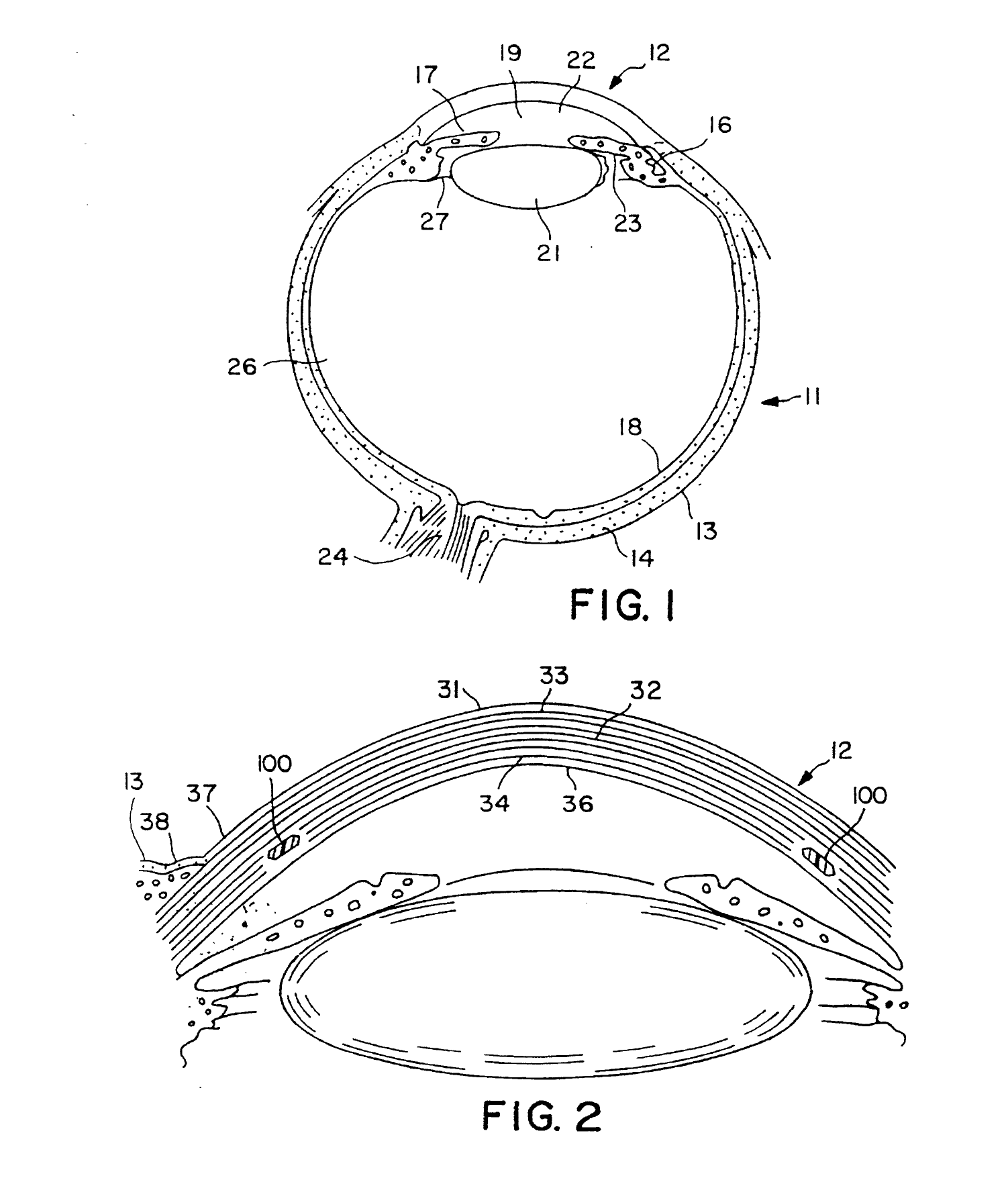

Referring to FIG. 2, a more detailed drawing of the anterior portion of the globe shows the various layers of cornea 12 comprising an epithelium 31. Epithelial cells on the surface thereof function to maintain transparency of cornea 12. These epithelial cells are rich in glycogen, enzymes and acetylcholine and their activity regulates the corneal corpuscles and controls the transport of water and electrolytes through the lamellae of the stroma 32 of cornea 12.

An anterior limiting lamina 33, referred to as Bowman's membrane or layer, is positioned between the epithelium 31 and stroma 32 of the cornea. Stroma 32 is comprised of lamella having bands of fibrils parallel to each other and crossing the whole of the cornea. While most of the fibrous bands are parallel to the surface, some are oblique, especially anteriorly. A posterior limiting lamina 34 is referred to as Descemet's membrane. It is a strong membrane sharply defined from stroma 32 and resistant to pathological processes of the cornea.

Endothelium 36 is the most posterior layer of the cornea and consists of a single layer of cells. Limbus 37 is the transition zone between the conjunctiva 38 and sclera 13 on the one hand and cornea 12 on the other.

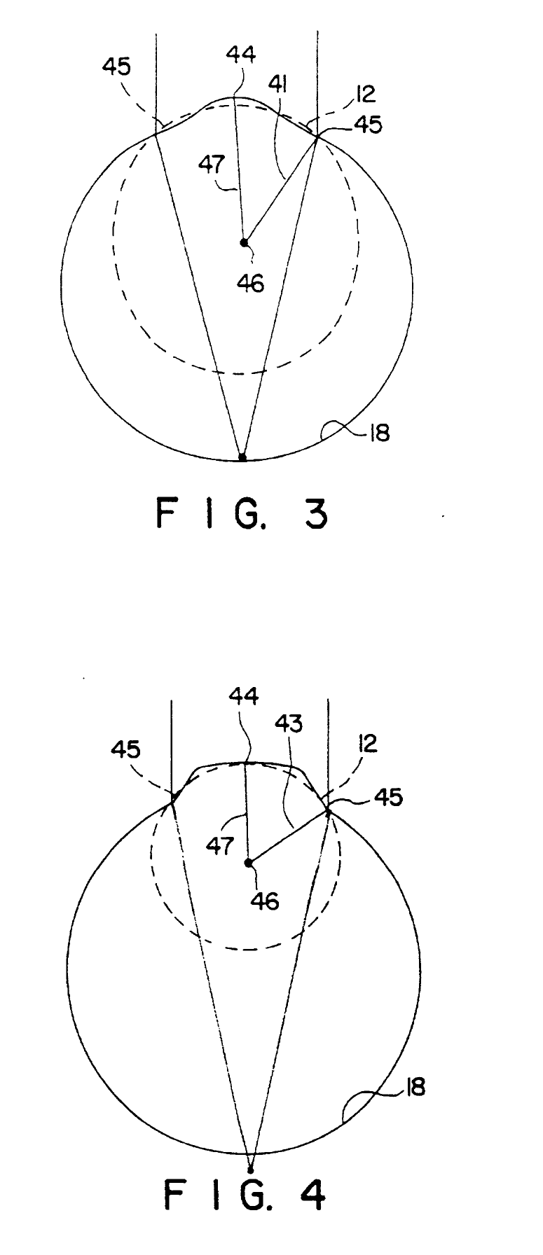

FIG. 3 shows the globe of the eye having a cornea 12 with an average spherical radius of curvature 41 and a positive aspheric shape. By “average spherical radius of curvature” we intend the radius of the circle defined by the points at the periphery 45 of the cornea near the limbus of the eye and having a center 46. By “positive aspheric shape” we mean that the distance 47 from that center 46 to the anterior center of the cornea is greater than the average spherical radius of curvature, that is, the anterior surface of the cornea flattens as it progresses from the center 44 to its periphery 45. As shown in FIG. 3, when parallel rays of light pass through the corneal surface, they are refracted by the corneal surfaces to converge eventually near retina 18 of the eye. The diagram of FIG. 3 discounts, for the purposes of this discussion, the refractive effect of the lens or other portions of the eye.

The eye depicted in FIG. 4 is hyperopic because the light rays from the periphery of the cornea refract into focus at a point behind the retinal surface. Further, the eye depicted in FIG. 4 has does not have the same aspheric shape as that shown in FIG. 3. Distance 47 from center 46 to the anterior surface of the cornea is about the same as or less than the average spherical radius of curvature 41 and the cornea does not flatten from center 44 to periphery 45 but rather plateaus or even dips at its center. If an intrastromal corneal ring with a flat mismatched cone angle, according to the present invention and as will be described in detail below, is implanted into the cornea shown in FIG. 4, the light rays refracted by the now steepened corneal surface will be refracted at a larger angle and thus converge at a more near point such as directly on the retina. Further, selection of an intrastromal corneal ring having a mismatched cone angle may allow for the eye to obtain a more positive aspheric shape similar to that shown in the FIG. 3 eye.

With the background discussion of FIGS. 1-4 in hand, it should be understood that the device and method of the present invention is for the adjustment of at least a portion of an annular chord of the cornea to decrease the radius of curvature and/or change the shape of the cornea in order to improve the vision of the eye. According to the present invention, the corneal geometry or refractive properties of the cornea are changed as a function of cone angle which is described in detail below.

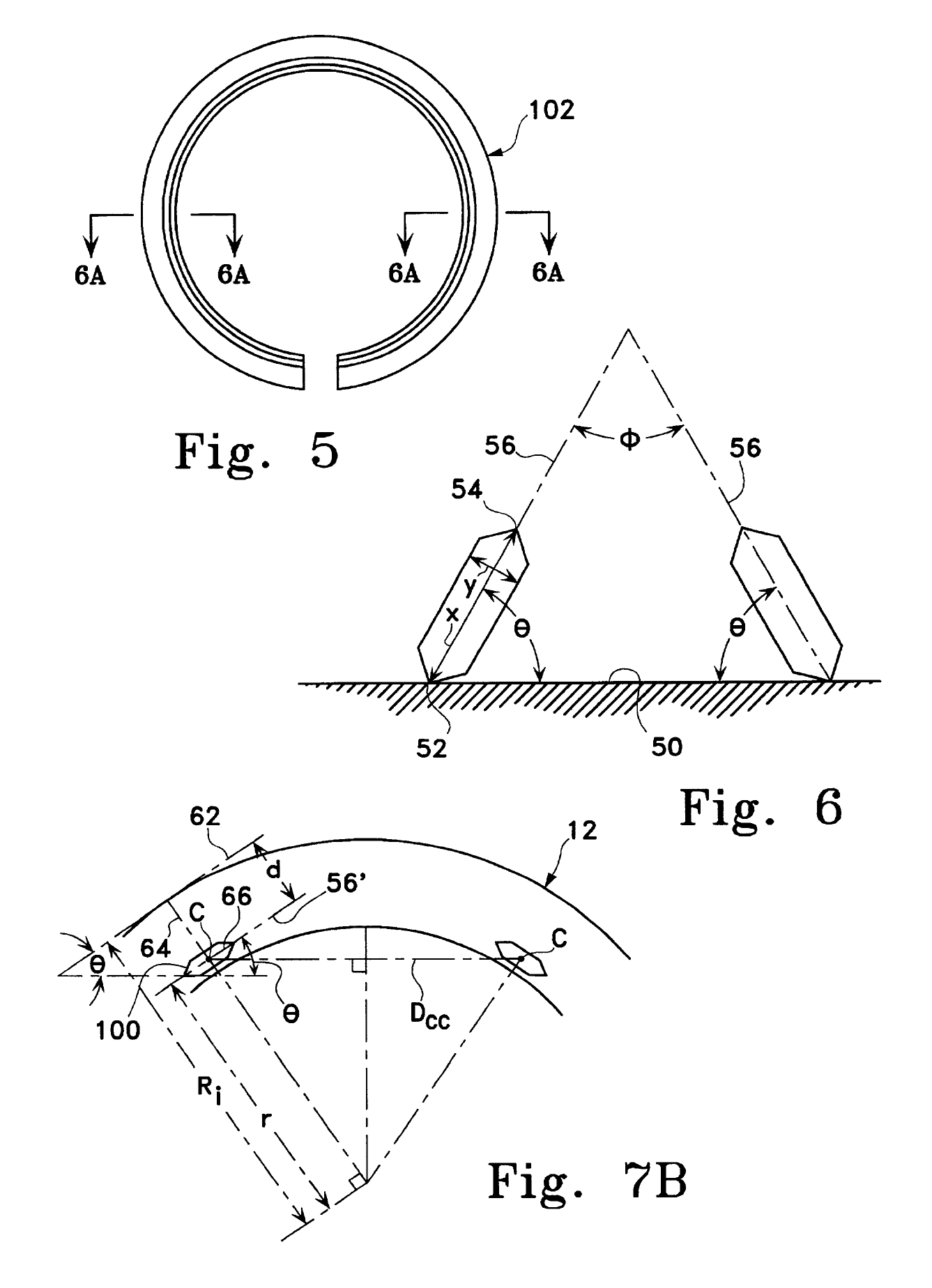

Referring to FIGS. 5 and 6, intrastromal corneal ring 102 is shown in accordance with the invention. Referring to FIG. 5, intrastromal corneal ring 102 is comprised of a generally circular member having split end portions (or open end portions). That is, intrastromal corneal ring 102 has a split ring configuration. In the preferred embodiments, the intrastromal corneal ring material has properties that render it physiologically compatible with the tissue of the cornea. An illustrative material is a plastic type material sold under the trade name of PERSPEX CQ™ (Imperial Chemical Company, England). However other biocompatible polymers including but not limited to Teflon, and polysulfones can be used.

Referring to FIG. 6, intrastromal corneal ring 102 has a hexagonal cross-sectional shape as illustrated in FIG. 6 where a radial transverse section is shown. In the illustrative embodiment, the cross-section may be referred to as one that cuts through the diameter of the intrastromal corneal ring and is at an angle of 90° to flat surface 50 upon which intrastromal corneal ring 102 is shown supported. As evident from the foregoing, one suitable cross-sectional shape for the intrastromal corneal rings of the present invention is the hexagonal shape shown in FIG. 6. This cross-section is generally dimensioned to be about 0.5 mm to 2.0 mm from point 52 to point 54 along its major axis which is designated with reference numeral 56. This dimension is designated with reference character “x”. The thickness of the intrastromal corneal ring, generally designated with reference character “y”, preferably is from about 0.05 mm to 0.60 mm in this embodiment.

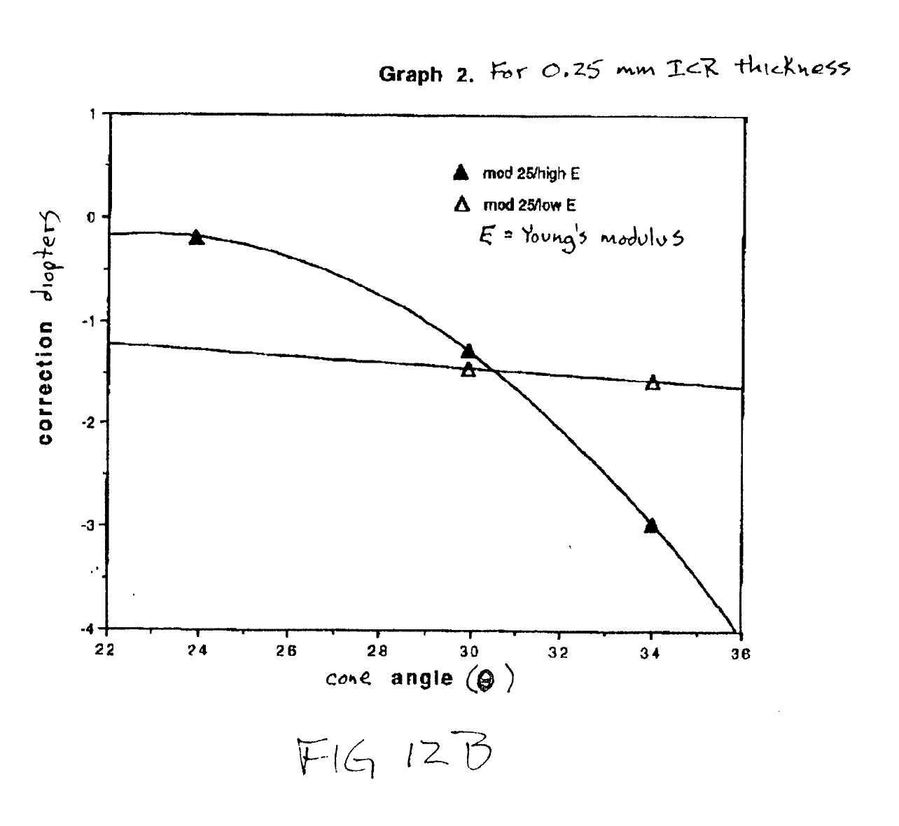

Although a particular intrastromal corneal ring configuration has been described above, rings of other cross-sectional shapes, including but not limited to ovoloid and rectangular shapes also may be used. Illustrative examples of generally ovoloid shapes are provided in FIGS. 10 and 11, which will be discussed in more detail below. It should also be understood that although a split ring is shown, continuous or closed rings can be used as well as other ring configurations.

Returning to FIG. 6, the cone angle θ of an intrastromal corneal ring is defined, for example, as the angle between the plane of the flat surface 50 that the intrastromal corneal ring rests on and a line drawn between points 52 of the cross-section that rests on the flat surface and point 54 on the cross-section that is farthest from the point where the intrastromal corneal ring rests on the flat surface. In other words, cone angle θ is the angle formed between the major axis of a radial, transverse cross-section (e.g., axis 56 as shown in FIG. 6) and surface 50. Alternatively, cone angle θ can be defined with reference to the angle formed by the intersection of major axes 56, angle φ, where θ=(180−θ)/2.

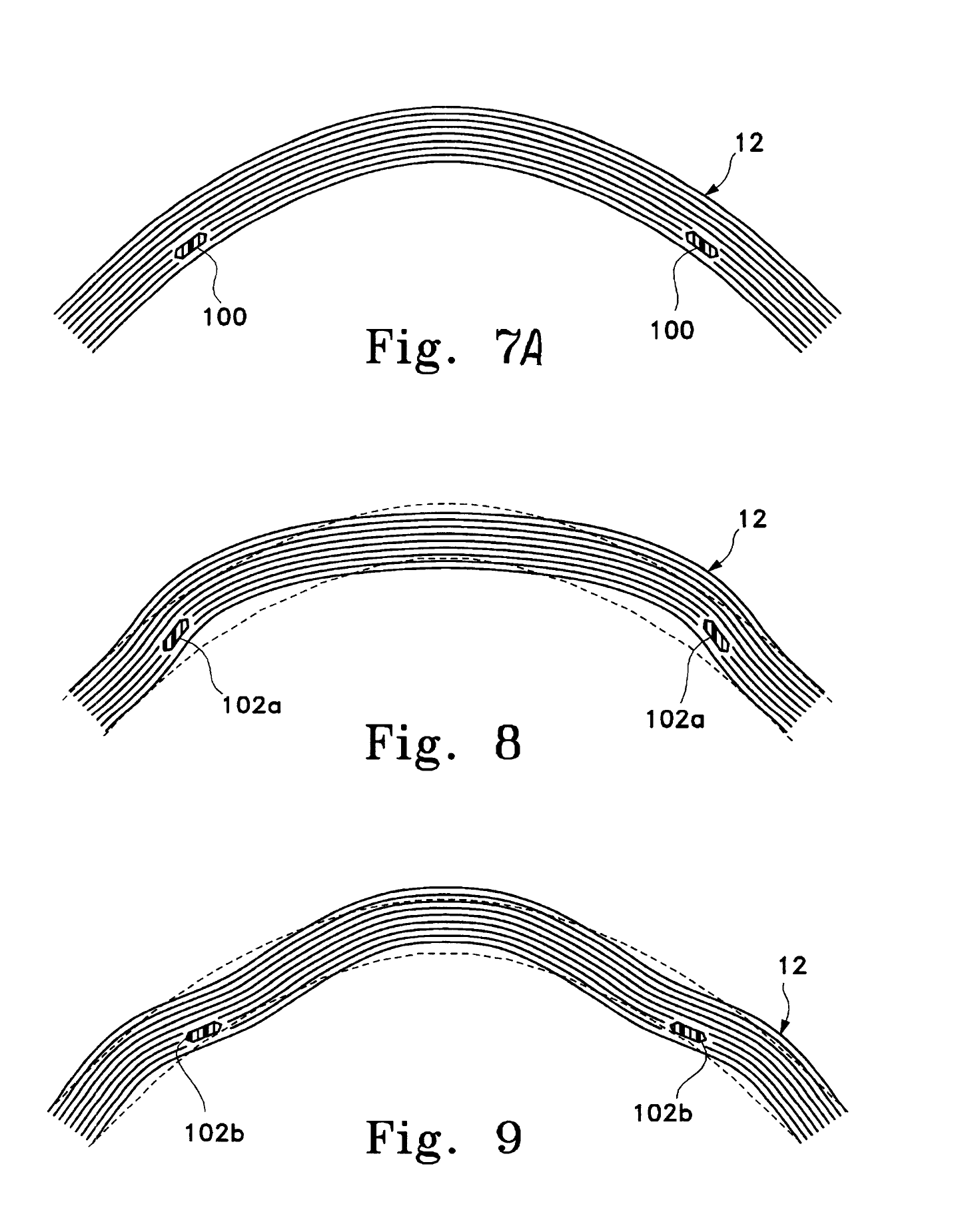

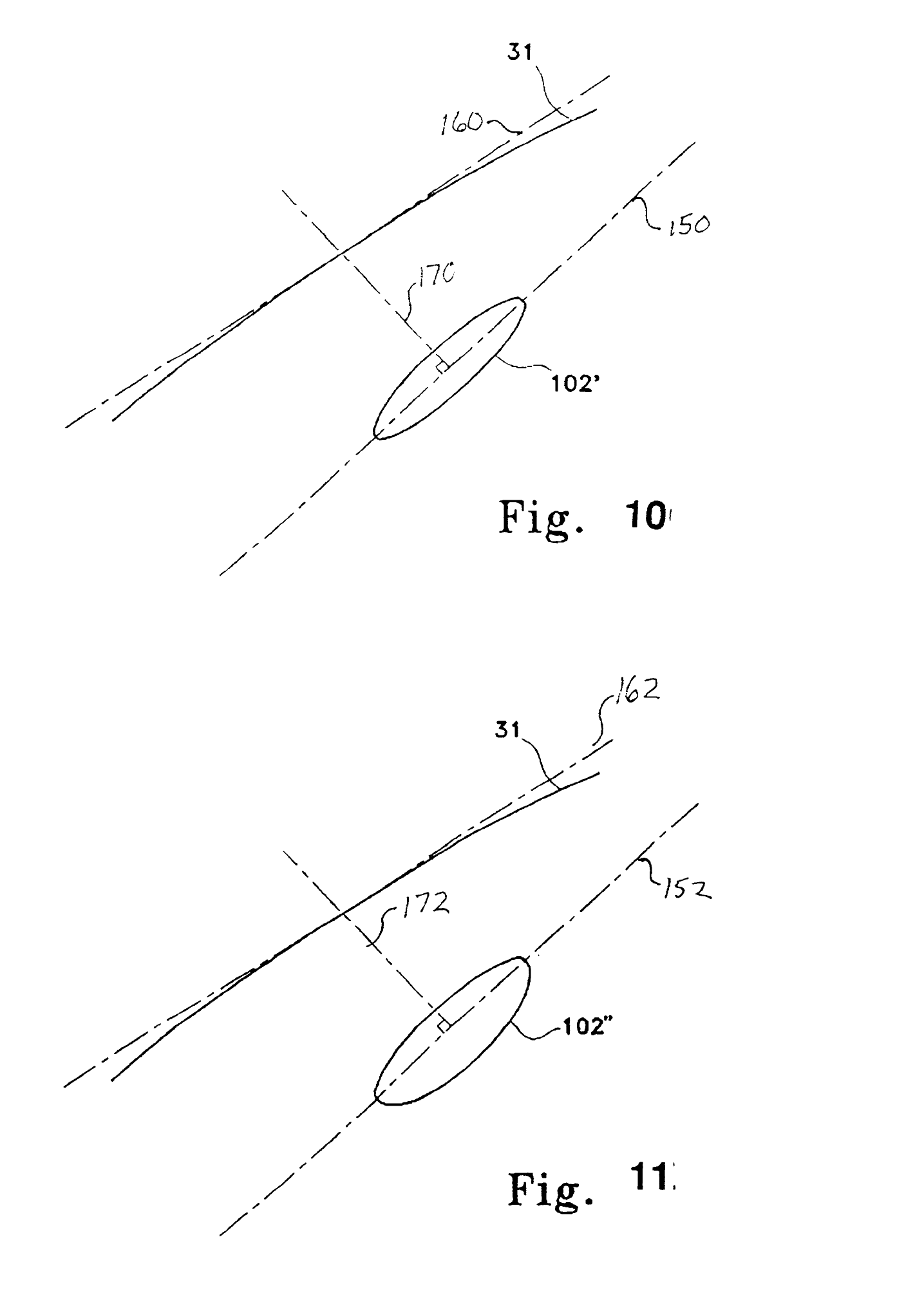

As discussed above, an important aspect of the invention is that the shape of the anterior corneal surface may be adjusted by using intrastromal corneal rings having mismatching “cone angles”. This is generally illustrated in FIGS. 7A, 8 and 9 where the effect of mismatching and matching cone angles is compared.

Referring to FIG. 7A, the effect of inserting an intrastromal corneal ring, such as intrastromal corneal ring 100 described above, having a cone angle matched to the corneal architecture prior to insertion is generally shown. A matched cone angle may generally be considered to be one that matches the angle of a particular line that is tangent to the anterior surface of the cornea. That tangent line is obtained by radially projecting the line that extends along the major axis of a radial, transverse cross-section of the intrastromal corneal ring (e.g., along the line indicating dimension x in FIG. 6) to the anterior corneal surface. A matching cone angle may vary according to changes in corneal shape and to the dimension of the intrastromal corneal ring used.

More specifically, a matching cone angle can be described as follows with reference to an imaginary intrastromal corneal ring superimposed at the insertion site prior to insertion of the intrastromal corneal ring. The major axis of substantially any transverse cross-section of the intrastromal corneal ring would be parallel to a line in the same plane as that major axis and tangent to the anterior surface of the cornea at the point where the line that bisects the major axis line, and is perpendicular thereto, intersects the anterior surface of the cornea. The major axis line is defined as the line which (1) extends along the major axis and (2) is bounded by the outer surface of the intrastromal corneal ring. Such a major axis, tangent line, bisecting line and major axis line are shown in FIG. 7B and designated with reference numerals 56, 62, 64 and 66, respectively.

In deriving an equation to calculate matching cone angles, applicants considered the variables listed below to be important in determining the corneal radius change, ΔR₀ₛ, achieved by implanting the ring in the cornea.

ΔR₀ₛ=f (Rᵢ, θ, t, d, Dᵢ, D_{cc}, E_{y}/where

Rᵢ=initial corneal radius of curvature (measured along the anterior corneal surface) (see, e.g., FIG. 7B)

θ=cone angle of the intrastromal corneal ring

t=intrastromal corneal ring thickness

d=depth of the intrastromal corneal ring in the cornea measured radially from the anterior corneal surface to the midpoint of a radial line, extending across the thickest or largest radial dimension (e.g., “y” in FIG. 6) of the radial, transverse section referenced above with respect to D_{cc} and shown, for example, in FIG. 7B where the depth is indicated with reference character “r”.

Dᵢ=limbus diameter

D_{cc}=diameter of intrastromal corneal ring (center to center, where each center is at the midpoint of the major axis line of a radial, transverse section of the intrastromal corneal ring. Such centers are shown in FIG. 7B, for example, where they are indicated with reference character “c”).

E_{y}32 Young's modulus for the intrastromal corneal ring

Oₛ=intrastromal corneal ring cross-sectional area shape factor

$$= f \frac{LXA}{( CXA )}$$

where LXA is the long axis of the radial, transverse cross-sectional area of the intrastromal corneal ring and CXA is the circumference of the radial, transverse cross-sectional area of the intrastromal corneal ring.

These variables are believed to be the dominant ones that will indicate how the intrastromal corneal ring will perform in changing corneal curvature or shape.

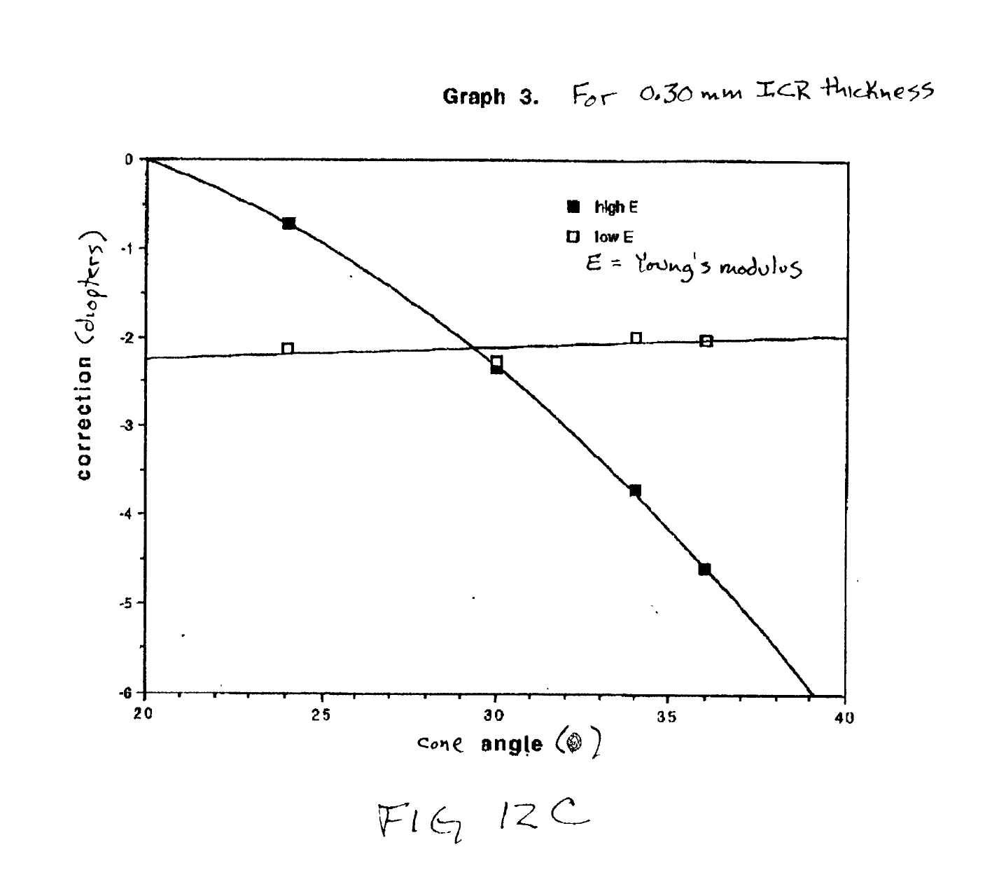

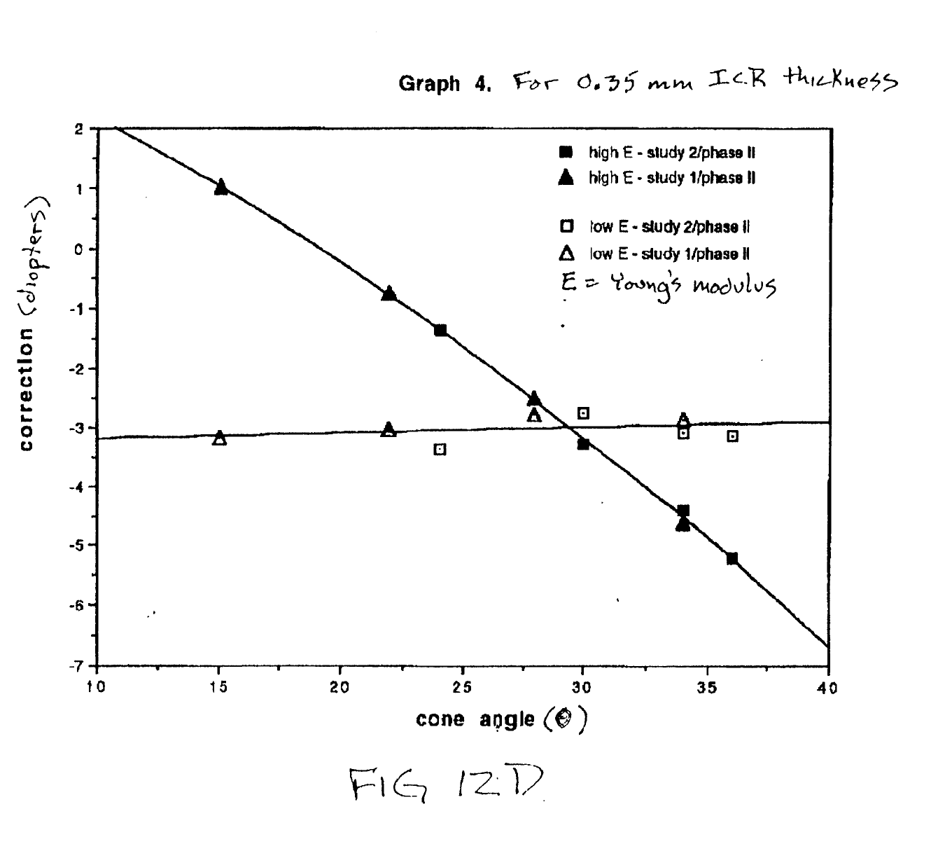

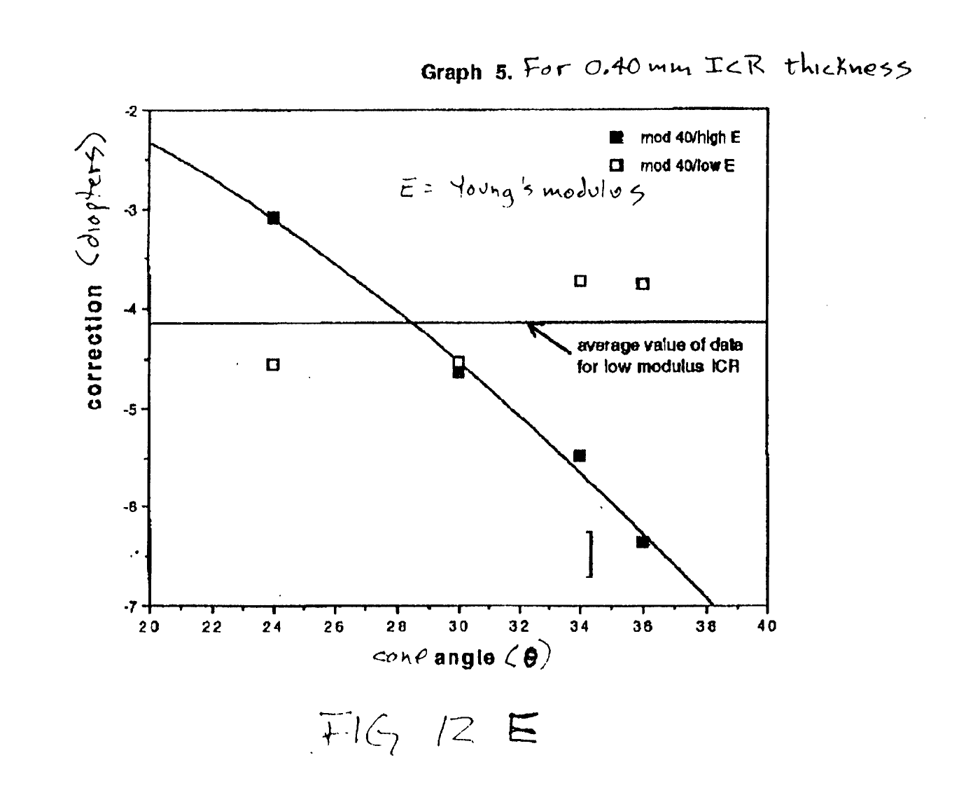

The graphs shown in FIGS. 12A, 12B, 12C, 12D and 12E show an example of fixing every variable constant with the exception of cone angle and Young's modulus at a given thickness. The point where the curves intersect generally corresponds to a matched cone angle. In general, with a high modulus, an increase in cone angle provides an increase in the minus correction which means an increase in the flattening of the cornea. By lowering the cone angle, one induces a positive correction which results in steepening the cornea. Correction is proportional to the radial curvature of the cornea. The correction values in the graphs correspond to 337.5/ΔRₜ. ΔRₜ is the expected radius of corneal curvature change induced by intrastromal corneal ring thickness independent of cone angle for a given transverse, cross-sectional shape of any particular design where the change is measured as the initial radius of corneal curvature (i.e., the radius of curvature of the cornea prior to intrastromal corneal ring implantation) minus the final radius of corneal curvature (i.e., the radius of curvature of the cornea after intrastromal corneal ring implantation).

Further examples indicating that increasing the cone angle increases refractive corrections are provided with respect to data obtained from tests conducted on human eye bank eyes. A cone angle of 25°, 34°and 40° provided refractive corrections of about −1.9, −2.7 and −6.0 diopters, respectively. Although the intrastromal corneal ring cone angles varied, each intrastromal corneal ring had a thickness (t) of about 0.30 mm and was inserted at a depth (d) in the cornea of about 0.42 mm.

Based on the variables listed above, the following equation, which defines a matching cone angle for a given D_{cc}, Rᵢ and d, was derived.

$$θ = {sin}^{- 1} \frac{{D}_{cc}}{2 ( {R}_{i} - d )} , where \qquad ( 1 )$$

θ, D_{cc}, Rᵢ and d are as defined above.

The following table provides matching cone angle values (θ) in degrees rounded to the nearest tenth of a degree for an intrastromal corneal ring implanted at a depth of 0.42 mm and for an initial corneal radius of curvature (Rᵢ) ranging from 7.6-7.9 mm (which is the typical range in the population) and center to center diameters (D_{cc}) ranging from 5.0-8.0 mm according to the above equation.

| | D_{cc} | | | | | | |
|---|---|---|---|---|---|---|---|
| Rᵢ | 5.0 | 5.5 | 6.0 | 6.5 | 7.0 | 7.5 | 8.0 |
| 7.6 | 20.4 | 22.5 | 24.7 | 26.9 | 29.2 | 31.5 | 33.9 |
| 7.7 | 20.1 | 22.2 | 24.3 | 26.5 | 28.7 | 31.0 | 33.3 |
| 7.8 | 19.8 | 21.9 | 24.0 | 26.1 | 28.3 | 30.5 | 32.8 |
| 7.9 | 19.5 | 21.6 | 23.6 | 25.8 | 27.9 | 30.1 | 32.3 |

The above calculations are merely exemplary and not intended to limit the invention. For example, the implantation depth “d” may range from about 0.10-0.50 mm, even though a value of 0.42 mm is used throughout the above calculations for purposes of example.

A mismatching cone angle is one that does not equal the cone angle described in equation (1) for a given D_{cc}, Rᵢ and d. Thus, for example, given a D_{cc} of 7.0 mm, an Rᵢ of 7.6 mm and a “d” of 0.42 mm, a mismatching cone angle would be any cone angle that is not equal to 24.1 degrees.

Another equation for describing a cone angle that is a matching cone angle is preferred when accounting for intrastromal corneal ring thicknesses above about 0.15 mm, which provides sufficient thickness to flatten the cornea independent of cone angle. According to this refined equation:

$$θ = {sin}^{- 1} \frac{{D}_{cc}}{2 [ ( {R}_{i} - d ) + \vert Δ {R}_{t} \vert ]} , where \qquad ( 2 )$$

ΔRₜ the expected radius of corneal curvature change induced by intrastromal corneal ring thickness independent of cone angle for a given transverse, cross-sectional shape of any particular intrastromal corneal ring design. Alternatively, ΔRₜ can be described as the initial radius of corneal curvature minus the final radius of corneal curvature as previously described above where ΔRₜ is determined based only on the change induced by the intrastromal corneal ring thickness independent of cone angle for a given radial, transverse cross-sectional shape of any intrastromal corneal ring.

Thus, when accounting for such thicknesses, a mismatching cone angle is one that does not equal the cone angle described in equation (2) for a given D_{cc}, Rᵢ, d and ΔRₜ.

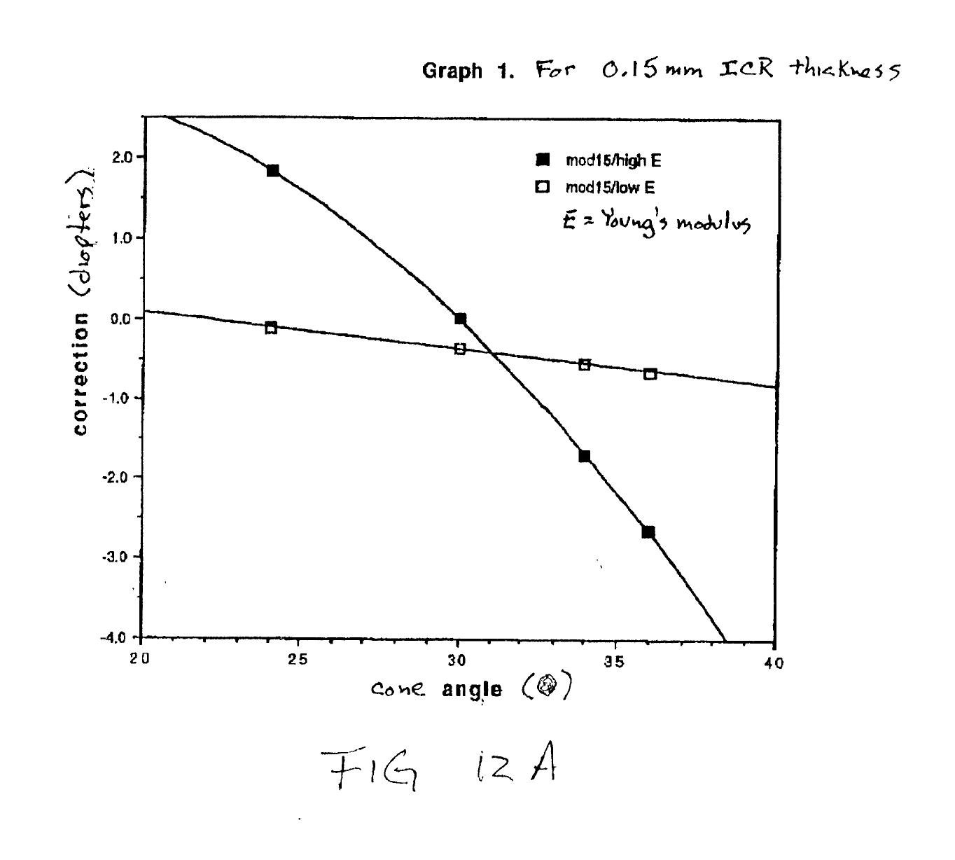

Returning to FIGS. 8 and 9, where intrastromal corneal rings having mismatched cone angles are shown, further principles of the invention will be described. Referring to FIG. 8, intrastromal corneal ring 102a is shown with a cone angle greater than that shown in FIG. 7. This twists adjacent portions of the cornea outward and flattens the central region of the cornea within the intrastromal corneal ring diameter as shown in the drawing. In contrast, FIG. 9 shows an intrastromal corneal ring 102b, which is the same as intrastromal corneal ring 102a with the exception that it its cone angle is less than that shown in FIG. 7, according to another embodiment of the invention. This cone angle effects a steepening of the corneal surface as shown in the drawing. In FIGS. 8 and 9 the phantom lines correspond to the outer lines of the cornea section of FIG. 7 and thus provide a reference for the corneal configuration changes shown in FIGS. 8 and 9.

Referring to FIGS. 10 and 11, the concept of a mismatching cone angle will be described in detail with reference to imaginary ovaloid intrastromal corneal rings (102′, 102″) superimposed at the insertion site prior to insertion of the cornea. As shown in the drawings, the major axis (axes 150 and 152 in FIGS. 10 and 11, respectively) of substantially any radial, transverse cross-section of the intrastromal corneal ring are not parallel to a line (lines 160 and 162 in FIGS. 10 and 11, respectively), which is in the same plane as the major axis and is tangent to the anterior surface of the cornea at the point where the line (line 170 and 172 in FIGS. 10 and 11, respectfully) that bisects the major axis line (defined as the line extending along the major axis and bounded by the outer surface of the intrastromal corneal ring) and is perpendicular thereto, intersects the anterior surface of the cornea. Alternatively, a mismatching cone angle may be defined with respect to one of equations (1) or (2) as described above.

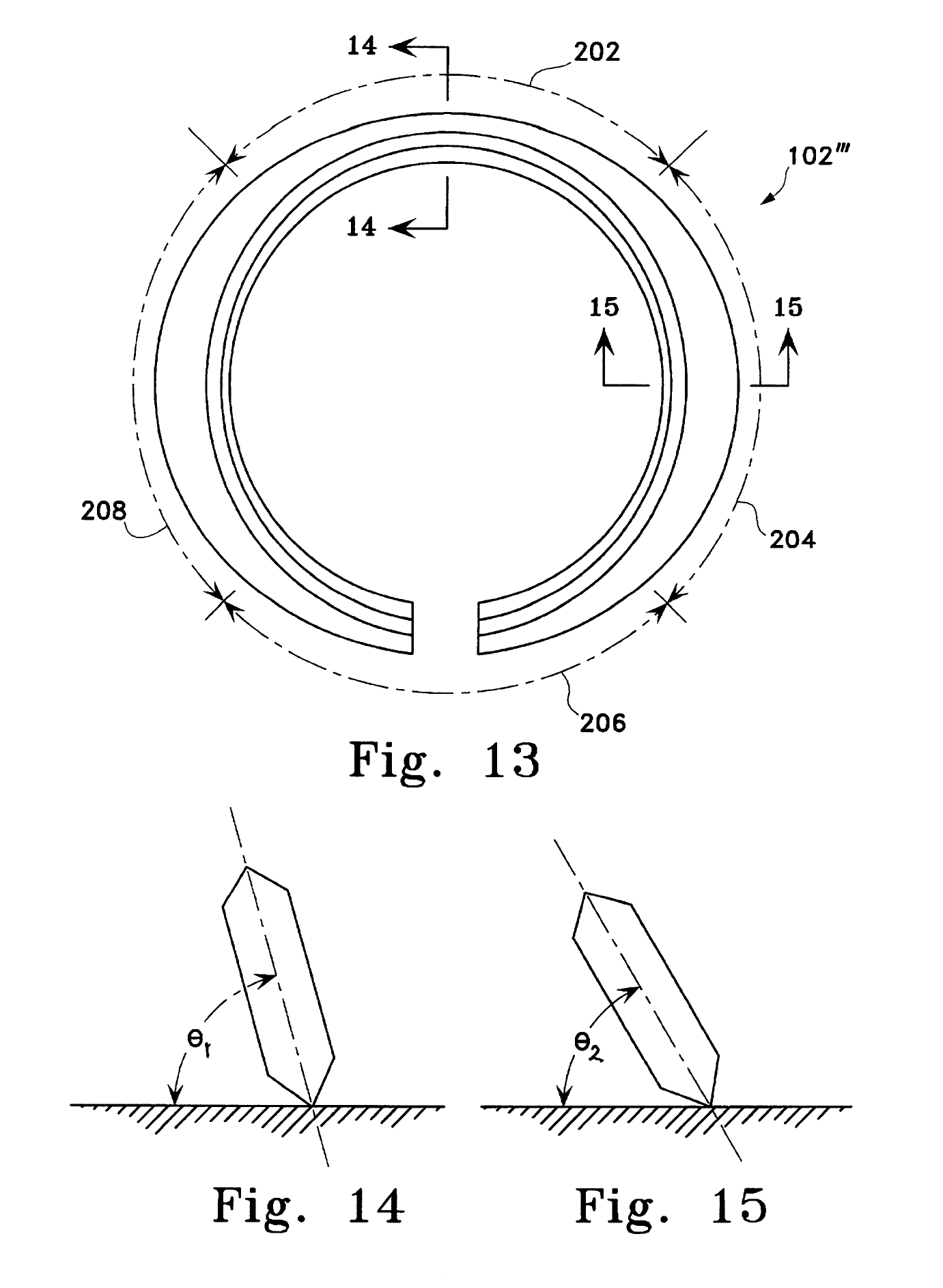

Referring to FIGS. 13-15, a further embodiment of the invention is shown. In this embodiment, intrastromal corneal ring 102′″, which preferably is constructed of materials as described above with respect to the example shown in FIGS. 5 and 6, is provided with multiple cone angles. This construction is particularly advantageous for treating astigmatism (or astigmatism concurrent with either myopia or hyperopia) where the corneal curvature varies in different meridians. In the embodiment shown in FIG. 13, the intrastromal corneal ring cone angle changes along the circumferential direction thereof. Generally speaking, the intrastromal corneal ring has a first circumferential region having a first cone angle and at least one other region having a cone angle that differs from said first cone angle.

In the example illustrated in FIG. 13, the intrastromal corneal ring has four circumferential regions with distinct cone angles. Circumferential region 202 has a first cone angle θ₁ as shown in FIG. 14. Circumferential region 202 is followed in the clockwise direction by a second circumferential region 204 having a second cone angle θ₂ (FIG. 15) that substantially differs from the first cone angle θ₁. That is, the cone angle varies enough for treating astigmatism (or astigmatism concurrent with either myopia or hyperopia) as discussed below. This variance typically may range from about 0° 10′ to about 20°.

Circumferential region 206 follows region 204 with a cone angle similar to that of region 202 and region 208, which follows region 206 in the clockwise direction has a cone angle similar to region 204. The cone angle transitions between regions may be abrupt or gradual depending on the desired change to the corneal shape. As in the intrastromal corneal rings described above, the intrastromal corneal ring 102′″ preferably is configured to substantially encircle the cornea after insertion. It should be noted, however, that although a particular multiple cone angle configuration has been shown, other configurations can be used. For example, a saddle shaped ring may be used as well as rings having more circumferential regions or circumferential regions having different shapes or dimensions than those shown. In addition, although a split ring configuration is shown, it should be understood that other configurations such as continuous or closed loop rings can be used.

As discussed above, this embodiment is particularly advantageous for treating astigmatism (or astigmatism concurrent with either myopia or hyperopia). Astigmatism is a condition that occurs when the parallel rays of light do not focus to a single point within the eye, but rather have a variable focus due to the fact that the corneal curvature varies in different meridians. According to the present invention, the cone angles in the multiple cone angle embodiment can be selected to change the shape of the cornea such that the light rays tend more to focus at a point.

The intrastromal corneal rings discussed above may be installed in the inner lamellar regions of the corneal stroma using known methods and devices. One preferred method and accompanying apparatus for implanting the intrastromal corneal rings is described in PCT/US93/03214 which is incorporated herein by reference in its entirety. In general, the intrastromal corneal ring is installed in the following manner: A small radial incision is made at the radius in which the ring is ultimately to be installed about the cornea. A dissector in the form of a split ring and having a point suitable for producing an interlamellar channel or tunnel in the corneal stroma is introduced through the small incision and rotated in such a fashion that a generally circular channel is formed completely about the cornea. The dissector is then rotated in the opposite direction to withdraw it from the tunnel thus formed. The intrastromal corneal ring is then introduced into the circular channel.

The above is a detailed description of particular embodiments of the invention. It is recognized that departures from the disclosed embodiments may be made within the scope of the invention and that obvious modifications will occur to a person skilled in the art. The full scope of the invention is set out in the claims that follow and their equivalents. Accordingly, the claims and specification should not be construed to unduly narrow the full scope of protection to which the invention is entitled.

We claim:

1. A method for changing the refractive characteristics of an eye comprising the steps of:
(a) providing a group of intrastromal corneal rings (ICRs) having different cone angles;
(b) determining an amount of corrective refraction desired;
(c) selecting an ICR from the group of ICRs based on the mount of corrective refraction determined in step (b); and
(d) inserting the ICR selected in step (c) into the cornea of the eye.

2. The method of claim 1 wherein a number of the ICRs provided in step (a) have different thicknesses.

3. The method of claim 1 wherein a number of the ICRs provided in step (a) have the same outer diameter but different cone angle.

4. The method of claim 1 further including the step of measuring the refractive correction after step (d) and if a desired amount of refractive correction has not been achieved, removing the inserted ICR and implanting a second ICR from the group and having a different cone angle.

5. The method of claim 4 wherein said second ICR is selected to have a cone angle greater than the cone angle of the ICR implanted in step (c) if the region of the cornea inside the ICR does not flatten by a desired amount.

6. The method of claim 4 wherein said second ICR is selected to have a cone angle less than the cone angle of the ICR implanted in step (c) if the region of the cornea inside the ICR does not steepen by the desired amount.

7. A method for changing the refractive characteristics of an eye comprising the steps of:
(a) inserting an intrastromal corneal ring (ICR) into the cornea of an eye at selected site; and
(b) selecting the ICR to have a cone angle such that if an imaginary corresponding ICR were superimposed at the insertion site prior to step (b), the major axis of substantially any transverse cross-section of the ICR would not be parallel to a line in the same plane as said axis and tangent to the anterior surface of the cornea at the point where the line that bisects said major axis line, defined as the line extending along said major axis and bounded by the outer surface of the ICR, and is perpendicular, thereto intersects the anterior surface of the cornea.

8. The method of claim 7 wherein the inserting step comprises inserting an ICR having a split ring configuration.

9. The method of claim 8 wherein the ICR is further configured so that it substantially encircles the cornea after insertion.

10. The method of claim 7 wherein the cone angle is selected to be between 0° and 50° and the ICR with a thickness of a out 0.05 and 0.60 mm.

15. In a modified cornea having an anterior surface and an intrastromal corneal ring (ICR) implanted therein, said ICR comprising a generally ring shaped member having a cone angle, an outer surface, a major axis for each radial, transverse cross-section thereof and a major axis line for each said cross-section, each major axis line defined as the line that extends along a respective major axis and is bounded by the outer surface of said generally ring shaped member, said cone angle being one that if an imaginary member having the same configuration, major axes and major axes lines as said generally ring shaped member were superimposed at an insertion site in said cornea prior to actual insertion of said generally ring shaped member, the major axis of substantially any radial, transverse cross-section of the imaginary member would not be parallel to a line in the same plane as that major axis and tangent to the anterior surface of the cornea at the point where a line, that bisects the major axis line and is perpendicular thereto, intersects the anterior surface of the cornea.

16. In a modified cornea having an intrastromal corneal ring (ICR) implanted therein, the ICR having a cone angle that effects a change in the configuration of the cornea from its preimplantation configuration as a result of exerting a twisting force on the corneal stroma about a portion of the ICR.

17. The cornea of claim 16 wherein the ICR has a split ring configuration.

18. The cornea of claim 16 wherein the ICR is configured to substantially encircle the cornea after insertion.

19. In intrastromal corneal ring (ICR) for insertion into the cornea of an eye, said ICR comprising a biocompatible ring having multiple cone angles.

20. The ICR of claim 19 wherein the ICR cone angle changes along the circumferential direction of the ICR.

21. The ICR of claim 19 wherein the ICR has a first circumferential region having a first cone angle and at least one other region having a cone angle that differs form said first cone angle.

22. The ICR of claim 19 wherein the ICR has a split ring configuration.

23. The ICR of claim 19 wherein the ICR is configured to substantially encircle the cornea after insertion.

24. An intrastromal corneal ring (ICR) having a cone angle and a center to center diameter (D_{cc}), for a given Rᵢ and d as defined below, the one angle being nonequal to the cone angle described according to the following equation:
$$θ = {sin}^{- 1} \frac{{D}_{cc}}{2 ( {R}_{i} - d )} , where$$
$$θ = cone angle ;$$
D_{cc}=diameter of the ICR (center to center, where each center is at the midpoint of the line that extends across a radial, transverse section of the ICR and along the major axis of that section;
Rᵢ=initial corneal radius of curvature; and
d=depth of the ICR in the cornea measured radially from the anterior surface of the cornea to the midpoint of a radial line extending across the largest radial dimension of the radial, transverse section referenced with respect to D_{cc} above) and bounded thereby.

25. An intrastromal corneal ring (ICR) having a cone angle and a center to center diameter (D_{cc}), for a given Rᵢ and d as defined below, the cone angle being nonequal to the cone angle described according to the following equation:
$$θ = {sin}^{- 1} \frac{{D}_{cc}}{2 [ ( {R}_{i} - d ) + \vert Δ {R}_{t} \vert ]} , where$$
$$θ = cone angle ;$$
D_{cc}=diameter of the ICR (center to center where each center is at the midpoint of the line that extends across a radial transverse section of the ICR and along the major axis of that section);
Rᵢ=initial corneal radius of curvature;
ΔRₜ=the initial radius of corneal curvature prior to ICR implantation minus the final radius of corneal curvature after ICR implantation where the ΔRₜ is determined based only on the change induced by the ICR thickness independent of cone angle for a given radial, transverse cross-sectional shape of any ICR; and
d=depth of the ICR in the cornea measured radially from the anterior surface of the cornea to the midpoint of a radial line extending across the largest radial dimension of the radial, transverse section referenced with respect to D_{cc} above) and bounded thereby.
